(19) United States
(12) Patent Application Publication
(10) Pub. No.: US 2023/0141195 A1
(43) Pub. Date: May 11, 2023

(54) LOW DEAD SPACE NEEDLE HUB

(71) Applicant: Juan Ernesto De la Puente DE LA PUENTE, Ciudad de Mexico (MX)

(72) Inventor: Juan Ernesto De la Puente DE LA PUENTE, Ciudad de Mexico (MX)

(21) Appl. No.: 17/645,394

(22) Filed: Dec. 21, 2021

(30) Foreign Application Priority Data
Nov. 5, 2021 (MX) MX/A/2021/013570

(51) Int. Cl. A61M 5/34; A61M 5/32

(52) U.S. Cl. CPC A61M 5/344; A61M 5/3293

(57) ABSTRACT

The present invention relates to a low dead space hub and a needle coupled to said hub, the dead space of which has a storage capacity of less than about 0.0190 milliliters.

### SUBJECT MATTER OF THE INVENTION

The present description relates generally to the field of medical needles for the administration of fluids, and more particularly to allow dead space needle hub that minimizes fluid waste.

### BACKGROUND

The term “dead space” refers to the space created by the coupling recess between a needle assembly and a syringe. In Luer-based syringes, the volume of the lumen of the nozzle plus the front cavity of the hub causes dead space. Conventional hypodermic needle assemblies waste a percentage of medication due to dead space associated with the volume of the lumen of the nozzle plus the front cavity of the hub; this waste is relevant when it comes to expensive medications. Also, significant amounts of drug can be trapped in the front of the hub cavity after an injection. It is contemplated that 0.10 milliliters of medicine can be trapped in the lumen of the nozzle plus the front part of the needle hub cavity; this value is regulated in the ISO 8537:2016 standard. For a 1.00 milliliter injection, 10% of the medicine is wasted. In addition, it is contemplated that the amount of wasted medicine can be up to 33% in case of administering 0.30 milliliters. A significant portion of the cost of giving an injection is commonly the expense of the drug. Reducing drug waste would significantly reduce healthcare costs.

Attempts have been made to reduce dead space by a conical sealing means. See, for example, U.S. Pat. Nos. 5,782,803, 5,902,271 and 5,902,277. However, these types of devices may have specialized tooling requirements that result in higher production costs. These devices may also require high precision manufacturing tolerances. Without being in accordance with these tolerances, disadvantageously the result is a large increase in dead space.

Therefore, there is a need in the art for a low dead space needle hub that effectively minimizes fluid waste.

### BRIEF DESCRIPTION OF THE INVENTION

The present invention relates to a hub for use in a syringe comprising a body with a proximal end and a distal end, said proximal end is adapted to couple with a syringe while the distal end is adapted to couple with a needle; wherein a needle holder with an interior channel is located inside the body, said needle holder extends beyond the proximal end of the hub preventing the solution from being trapped in the lumen of the syringe nozzle; and said needle holder has at least one sealing ring that is located on an external surface of the needle holder, said at least one sealing ring prevents the solution to be dispensed from being directed towards an inner chamber arranged between the needle holder and the body of the hub.

### BRIEF DESCRIPTION OF THE FIGURES

The accompanying Figures are included to provide further illustration and understanding of the various aspects and embodiments, and are incorporated into and constitute a part of this description.

The objects and features of the present description, which are believed to be new, are pointed out with particularity in the appended claims. The present description, as well as its organization and mode of operation, together with additional objectives and advantages, may be better understood by reference to the following description, taken in connection with the accompanying figures, wherein:

FIG. 1 is a cross-sectional view of a low dead space hub of the present invention mounted on a state of the art Luer Lock syringe.

FIG. 2 is a side view of the low dead space hub.

FIG. 3 is a front view of the low dead space hub.

FIG. 4 is a cross-sectional view of the low dead space hub of the present invention mounted on a state of the art Luer slip syringe.

### DETAILED DESCRIPTION OF THE INVENTION

The following detailed description is exemplary only and is not intended to limit the disclosed embodiments or the application and uses of the described embodiments. As used herein, the word “exemplary” or “illustrative” means “serving as an example, instance, or illustration.” Any implementation described herein as “exemplary” or “illustrative” should not necessarily be construed as preferred or advantageous over other implementations. All of the implementations described below are exemplary implementations provided to enable those skilled in the art to make or use the embodiments of the disclosure and are not intended to limit the scope of the disclosure. For the purposes of the present description, the terms “upper”, “lower”, “left”, “posterior”, “right”, “front”, “vertical”, “horizontal” and their derivatives will refer to the invention as oriented in the figures. Furthermore, there is no intention to be subject to any explicit or implicit theory presented in the technical field above, background, brief summary or the following detailed description. It should also be understood that the specific devices and processes illustrated in the accompanying drawings, and described in the following specification, are simply exemplary embodiments of the inventive concepts defined in the attached claims. Therefore, the specific dimensions and other physical characteristics related to the embodiments described herein should not be considered as limiting, unless the claims expressly state otherwise.

The exemplary needle assembly modalities described are discussed in terms of the administration of fluids to and/or from a subject, and more particularly, in terms of needle assemblies that minimize drug waste during the administration of an injection. The present description is considered to find application in the injection of preventive drugs, drugs to be administered by intradermal, subcutaneous, intramuscular or intravenous routes, as well as in injections used during procedures related to phlebotomy, dental, orthopedic, digestive, intestinal, urinary, veterinary, etc., to a subject.

In the discussion that follows, the term “close” or “proximal” will refer to the portion of a structure that is closest to the healthcare professional or physician, while the term “distant” or “distal” will refer to the portion that is farthest from the physician. As used herein, the term “subject” refers to a patient receiving injections from a syringe. In accordance with the present description, the term “physician” refers to an individual who administers an injection, installing or removing the needle assembly in or from a syringe, and may include support personnel.

For the purposes of the present invention it should be considered that the use of the term “approximately” provides a certain additional range. The term is defined as follows. The additional range provided by the term is ±10%. By way of example, but not limiting, if it says “approximately 40 cm”, the exact range is between 36 to 44 cm.

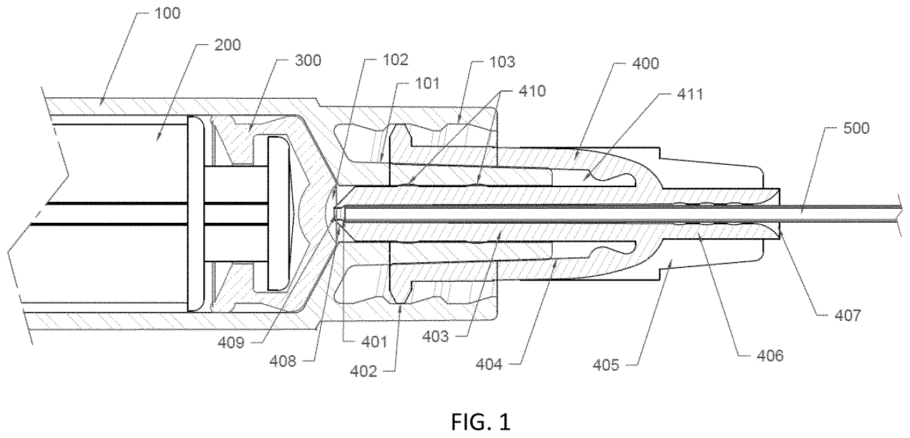

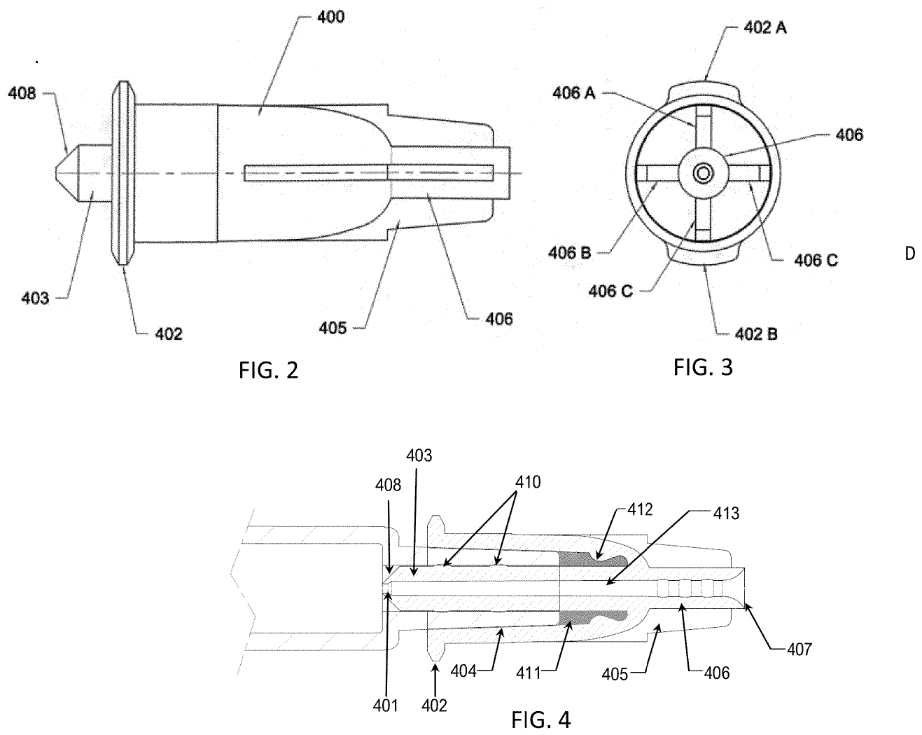

The low dead space needle assembly of the present invention is illustrated coupled with a first syringe (100) in FIG. 1, said first syringe (100) is a syringe with a Luer Lock cone (101), a plunger (200) and a piston (300); it should be mentioned that this first syringe (100) is a syringe known from the state of the art where the piston (300) is conical and has a cylinder cord (103). In a further embodiment, another type of Luer cone syringe can be used, such as a Luer slip cone syringe.

A hub (400) comprising a body with a proximal end and a distal end, said proximal end is adapted to couple with the first syringe (100) with a Luer connection (101) of the first syringe (100) while the distal end of said hub (400) is adapted to couple with a needle (500).

Inside the body of the hub (400) a needle holder (403) with an inner channel (413) is located, said needle holder (403) extends beyond the proximal end of the hub (400) at least one sealing ring (410) that is located on an external surface of the central needle holder (403); and an inner chamber (411) disposed between the needle holder (403) and the hub body (400); the number of sealing rings (410) can be from one to four. Said needle holder (403) comprises a cylindrical body, in such a way that the external surface of said cylindrical body is smooth and uniform.

The function of the at least one sealing ring (410) is to prevent the passage of a fluid to be dispensed from the syringe into the inner chamber (411), in which up to approximately 0.0185 ml of fluid can be stored, causing significant waste thereof.

The needle (500) is coupled at the distal end of the hub (400) to the inner channel (413) through a main assembly hole (407). It should be mentioned that said main assembly hole (407) has an internal bevel, such that the main assembly hole (407) has a larger initial diameter than a final diameter. Likewise, at the proximal end of the inner channel (413) a needle limit (401) is located, so that the needle (500) extends throughout the entire inner channel (413). It should be mentioned that the length of the inner channel (413) is between 90-98% of the length of said needle holder (403). Thus, the residual due to the dead space (102) in this area, plus the interior of the needle, plus the dead space generated in the inner cavity (411) is reduced by approximately 80% depending on the gauge and length of the needle compared to the current state of the art, such that the dead space (102) of the present invention plus the interior of the needle stores less than approximately 0.0190 milliliters compared to conventional needles that waste up to 0.10 milliliters.

Inside said inner channel (413) is at least one needle coupling cavity (406), said at least one needle coupling cavity (406) comprises at least one annular groove to be filled with glue and make contact with the needle (500) and thus prevent said needle (500) from disengaging.

The hub (400) has an external surface, on said external surface at a distal end is at least one pair of side parts (405); preferably, the number of side parts is four (406A, 406B, 406C, 406D) and said side parts (405) are arranged diametrically opposite each other.

The hub (400) has at its proximal end at least one flange for mounting (402) with rope; preferably, the number of cord mounting flanges (402) is two (402A, 402B), said two cord mounting flanges (402A, 402B) are arranged diametrically opposite each other.

Additionally, the hub (400) has an internal surface (404), at the end of said internal surface (404) there are molding releases (412), which serve to unmold the hub at the time of its manufacture.

The component parts of the needle assembly are manufactured from materials suitable for drug injections, such as, for example, polymeric materials or metals, such as stainless steel, depending on the particular medical needle application and/or the physicians preference. Semi-rigid and rigid polymeric materials are contemplated for fabrication, as well as elastic materials, such as medical grade molded polypropylene. However, a person skilled in the art will realize that other materials and manufacturing methods suitable for assembly and manufacture, in accordance with the present description, would also be suitable.

A person skilled in the art can modify the structure described herein. However, it should be noted that this description relates to preferred embodiments of the invention, and is provided for illustrative purposes only, and should not be understood as limiting the invention. All obvious modifications in the spirit of the invention, such as changes in the shape, material, and dimensions of the elements that make up the invention, should be considered within the scope of the attached claims.

The invention has been described in an illustrative manner and it should be understood that the terminology, which has been used herein, is intended to correspond to the nature of the words of the description rather than to provide a limitation.

Obviously, many modifications and variations of the present invention are possible in light of the above teachings. Therefore, it should be understood that within the scope of the invention described, the invention may be practiced otherwise than specifically described.

1. A hub for use in a syringe comprising a body with a proximal end and a distal end, said proximal end is adapted to couple with a syringe while the distal end is adapted to couple with a needle;
wherein a needle holder with an inner channel is inside the body, said needle holder extends beyond the proximal end of the hub preventing a solution to be dispensed from being trapped in a nozzle of the syringe; and
said needle holder has at least one sealing ring which is located on an external surface of the needle holder, said at least one sealing ring prevents the solution to be dispensed from directing towards an inner chamber arranged between the needle holder and the body of the hub.

2. The hub according to claim 1, wherein the needle holder comprises a cylindrical body and wherein the inner channel comprises a water boundary at a proximal end.

3. The hub according to claim 1, wherein the needle holder has a guide cone at its outer distal end.

4. The hub according to claim 1, wherein said hub has a main assembly hole at the distal end.

5. The hub according to claim 4, wherein said main assembly hole has an internal bevel, such that the main assembly hole has a larger initial diameter than a final diameter.

6. The hub according to claim 5, wherein within said main assembly hole is located at least one needle coupling cavity.

7. The hub according to claim 1, wherein the number of sealing rings is from one to four.

8. The hub according to claim 1, wherein the hub body has an internal surface, on said internal surface there is a mold release.

9. A syringe comprising:
a syringe body having a cone, a plunger, and a piston;
a hub according to claim 1, which couples with the cone of the syringe body.
